(19) United States
(12) Patent Application Publication
(10) Pub. No.: US 2007/0238121 A1
(43) Pub. Date: Oct. 11, 2007

(54) Methods and apparatuses for comparative genomic microarray analysis

(76) Inventors: Blake C. Ballif, Greenacres, WA (US); Lisa G. Shaffer, Colbert, WA (US); Bassem A. Bejjani, Colbert, WA (US)

Correspondence Address: Scott R. Maynard, Suite 1000, 2020 Main Street, Irvine, CA, 92614, US

(21) Appl. No.: 11/731,424

(22) Filed: Mar. 30, 2007

Related U.S. Application Data
(60) Provisional application No. 60/790,323, filed on Apr. 6, 2006.

(51) Int. Cl. C12Q 1/68; C12M 3/00

(52) U.S. Cl. 435/6; 435/287.2

(57) ABSTRACT

Control nucleic acids and their method of use to simultaneously test for numerous genetic alterations that involve an unbalanced arrangement of chromosomes. One implementation increases reliability and accuracy by adding additional nucleic acid to test and/or reference samples. Clones representing segments insensitive to chromosomal rearrangements are placed in non-adjacent target areas of a microarray to avoid interfering hybridization reactions.

This patent is a continuation of a provisional patent application entitled Methods and Apparatuses for Comparative Genomic Microarray Analysis filed on Apr. 6, 2006.

### TECHNICAL FIELD

The subject matter relates generally to molecular biology and more specifically to methods and apparatuses for achieving precision genetic diagnoses.

### BACKGROUND

Chromosome analysis is an important component in diagnosing congenital anomalies that cause physical anomalies and developmental delay. Cytogenetic imbalance results in DNA copy-number changes and alteration in gene dosage in the altered chromosomal segment(s). These changes may result in abnormal clinical phenotypes. Such chromosomal aberrations are conventionally detected by a variety of methods, each with distinct advantages and disadvantages. Routine cytogenetic analysis by GTG banding can achieve resolution sufficient to detect aneuploidy and structural rearrangements of base-pair sequences greater than five megabases (Mb) but cannot reliably identify abnormalities less than five Mb.

More subtle genetic alterations or those involving regions that are difficult to visualize may be undetectable by conventional cytogenetic techniques (e.g., including most microdeletion syndromes and exchanges of similarly banded segments that lead to cryptic translocations). Fluorescence in situ hybridization (FISH) was developed to probe individual chromosomal loci at a resolution equal to the size of the probe used in the assay (e.g., 35-200 kilobases (Kb)). Only a few loci may be examined at a time, however, and FISH can usually only be performed in a limited manner based on phenotype. Thus, single locus FISH is not an appropriate screening tool for the analysis of more than a few loci at a time.

Additional molecular cytogenetic techniques were developed to overcome limitations in FISH and GTG banding. Comparative genomic hybridization (CGH) was developed to identify chromosomal imbalance without the need for phenotypic information, circumventing multiple FISH experiments. CGH provides genome-wide screening of genetic sequence alterations by comparing differentially labeled test and control samples of genomic DNA. The resolution of the technique, however, is still limited to approximately 5-10 Mb because metaphase chromosomes are used as the targets for analysis.

To substantially increase the resolution, CGH-based microarrays for performing “array CGH” were developed. Array CGH is a high-resolution, comprehensive method for detecting both genome-wide and chromosome-specific copy-number imbalances. Array CGH typically uses large-insert clones (such as bacterial artificial chromosomes, “BACs”) as the target for analysis rather than metaphase chromosomes. As a consequence, the resolution of the array is limited only by the size of the insert used and the physical distance in the human genome between clones that are selected for the array.

CGH microarrays have been successfully constructed to test many parts of the human genome. In 2001, a whole-genome array was constructed using approximately 2400 BAC clones to scan for genome-wide copy-number alterations. An array covering some of the telomeric regions of the human genome has also been developed. Individual chromosomal regions have also provided good targets for array CGH. For example, in 2003 a microarray was designed to cover much of the most distal 10.5 Mb of chromosome location 1p36 to study subjects with monosomy 1p36. In 2003, an array was constructed based on chromosome 18 to study patients with congenital anal atresia. Microarrays have also been developed to test parts of chromosomes 20 and 22.

In a clinical setting, however, a conventional whole-genome approach to array CGH may cause erroneous test results that are due to undesirable polymorphisms, which are usually abundantly represented in this approach. Data from sub-telomere FISH analysis, for example, reveal many telomeric alterations that possess no clinical significance.

It is estimated that about 35% of clones that are currently available from the public and private databases either map to the wrong location, map to more than one location in the genome, represent polymorphic areas of the genome, or contain repetitive sequences that may interfere with hybridization. Using random clones from the databases would result in a clinical test that has more than a 35% probability of error and that is of dubious utility. Thus, “whole genome arrays” are not appropriate for clinical applications. The adoption of such “whole genome” arrays for use in clinical diagnostics may be unwise, not only leading to many false positive diagnoses that necessitate expensive follow-up confirmatory tests by FISH or other methods, but also additional blood draws from unaffected relatives of the patient to determine possible segregation of genetic deletions, duplications, or polymorphisms; not to mention unnecessary anxiety for the family of a person being tested. In the “whole genome” approach, hybridization results for single clones that show dosage difference require careful examination and each clinical case may require all the time and expense of a mini-research project. Thus, genome-wide “dense” arrays that are conventionally available for research use are not appropriate, relevant, or efficient in a clinical setting. There exists a need for a clinically useful diagnostic array that provides reliability, that accurately detects chromosome abnormalities such as aneuploidies, and that provides interpretable results with an acceptable degree of precision.

Approximately 10%-15% of all clinically recognized pregnancies end in miscarriage, most of which occur in the first trimester. Of these first-trimester miscarriages, about 50% are due to fetal chromosome abnormalities (Hassold et al. 1980), the majority of which (86%) arise from aneuploidies, including trisomies, monosomies, and polyploidies (triploidy or tetraploidy). Cytogenetic analysis of tissue from spontaneous abortions provides valuable insights into the cause of miscarriage, helps determine recurrence-risk estimates for subsequent pregnancies, and eliminates further costly testing. Currently, routine cytogenetic analysis relies on the successful culture of fetal tissue and preparation of metaphase cells, a well-established methodology in clinical cytogenetics laboratories. However, diagnosis in product of conception (POC) samples is often hindered by a relatively high (10%-40%) rate of tissue-culture failure (Lomax et al. 2000) and the suboptimal quality of chromosome preparations. In addition, selective overgrowth of maternally derived cells can occur, thereby erroneously yielding a normal karyotype despite possible underlying fetal chromosomal abnormalities (Bell et al. 1999).

Presently there exists a need to develop novel CGH arrays and controls useful for providing accurate and reliable diagnosis of aneuploidies in biological samples.

### SUMMARY

The subject matter described herein can be used in various fields to greatly improve the accuracy and reliability of nucleic acid analyses, chromosome mapping, and genetic testing of suspected genetic conditions. In one implementation, aspects of the subject matter are incorporated into the construction of a diagnostic array (“array”), such as a temporal array, or a spatial array of tests used, for example, in comparative genomic hybridization (CGH) microarrays. An exemplary high-availability diagnostic array for testing chromosomal loci associated with human disease and constructed according to aspects of the subject matter may use one or more exemplary features, including: selective screening of genetic loci, reliable coverage of the selected loci, strategic placement of control reference clones, strategic nonrandom distribution of the clones on the microarray, and/or redundant sub-arrays for comparison and dependability.

The invention provides a novel apparatus and methods for the detection of aneuploidies. In one embodiment of the invention an array for the detection of aneuploidy is disclosed that comprises a first plurality of nucleic acid segments corresponding to a first set of chromosomal loci wherein each chromosomal locus is capable of genetic alteration indicative of aneuploidy, and a second plurality of nucleic acid segments corresponding to a second set of chromosomal loci that are not associated with the aneuploidy being detected, wherein each of the first and second plurality of nucleic acid segments represents a portion of the base-pair sequence of a chromosomal locus; wherein each nucleic acid segment is immobilized to a discrete and known spot on a substrate surface to form an array of nucleic acids, wherein the nucleic acid segments representing chromosomal loci that are adjacent on a native chromosome are placed in non-adjacent target areas of the array.

The selected chromosomal loci are capable of genetic alteration indicative of aneuploidies. In two embodiments of the invention, the aneuploidies to be diagnosed include any imbalanced sex chromosomes (e.g., Turner syndrome (45,X), 47,XXX, 45,X/46,XY, 46,XX/46,XY, 47,XYY, 47,XXY, 46,XY/47,XYY, 46,XX/47,XX,del(Yp)).

In one embodiment of the invention at least some of the segments of the base-pair sequence overlap each other and the multiple clones assigned to represent the chromosomal locus represent the overlapping segments. In yet another embodiment multiple clones that remain constant regardless of genetic alterations of a chromosomal locus are located on the array and flank that chromosomal diagnostic locus. Another aspect of the invention is that the second set of chromosomal loci is obtained from across the human genome, including telomeric, pericentromeric and pseudoautosomal chromosomal loci. In one embodiment of the invention, the nucleic acid segments are derived from one of a bacterial artificial chromosome, a yeast artificial chromosome, a P1-derived artificial chromosome, a cosmid, a plasmid, a fosmid, a piece of cDNA, or a synthetic oligonucleotide.

In one embodiment of the invention, the second set of nucleic acids comprises nucleic acids derived from a non-human source (e.g., Drosophila, yeast or E. coli).

In one embodiment of the invention a set of controls is described for use in comparative genomic hybridization analysis wherein the nucleic acids are derived from a naturally occurring sample that comprises a known aneuploidy (e.g., Turner syndrome and 45,X, 47,XXX, Klinefelter syndrome and 47,XXY, or 47,XYY).

In yet another embodiment a set of control nucleic acids is comprised of a mixture of known amounts of cloned nucleic acid segments corresponding to a plurality of genetic material derived from a biological sample. The biological sample can be human or non-human (e.g., Drosophila, yeast, or E. coli).

In one aspect of the invention, the set of control nucleic acids for use in comparative genomic hybridization analysis comprises a first group of a known amount of nucleic acid segments added to a test sample and a second group of a known amount of substantially identical nucleic acid segments added to a reference sample. In one embodiment the first and second group of nucleic acid segments is human. In an alternative embodiment first and second groups nucleic acid segments are non-human (e.g., Drosophila, yeast, and E. coli).

One aspect of the invention describes a method of comparative genomic hybridization analysis that comprises the following steps: (a) providing an array comprising a plurality of nucleic acid segments, wherein each nucleic acid segment is immobilized to a discrete and known spot on a substrate surface to form an array and the nucleic acid segments comprise a substantially complete first genome of a known mammalian karyotype; (b) providing a first sample, wherein the sample comprises a plurality of genomic nucleic acid segments comprising a substantially complete complement of a first genome labeled with a first detectable label; (c) providing a second sample, wherein the sample comprises a plurality of genomic nucleic acid labeled with a second detectable label, and the genomic nucleic acid sample comprises a substantially complete complement of genomic nucleic acid of a cell or a tissue sample, and the karyotype of the second sample comprises a known or suspected abnormal karyotype (d) contacting the samples with the array of step (a) under conditions wherein the nucleic acid in the samples can specifically hybridize to the genomic nucleic acid segments immobilized on the array; (e) measuring the amount of first and second detectable label on each spot after the contacting of step and (d) determining the karyotype of the first sample by comparative genomic hybridization.

In one embodiment of this invention the karyotype of the second sample comprises a known aneuploidy (e.g., Turner syndrome and 45,X, 47,XXX, Klinefelter syndrome and 47,XXY, 47,XYY).

In one embodiment of the method of this invention, the array comprises additional nucleic acid segments derived from a non-mammalian source (e.g., Drosophila, yeast, and E. coli).

In one embodiment of the method of this invention, the first and/or second sample additionally contains a known amount of nucleic acid from a non-mammalian source (e.g., Drosophila, yeast, and E. coli).

In one embodiment of the method of this invention, the first and second sample additionally contains a known amount of nucleic acid. This can be from a mammalian or a non-mammalian source (e.g, Drosophila, yeast, and E. coli). The known amount of nucleic acid added to the first and second sample can be used to generate a standard curve of ratios in a comparative genomic hybridization experiment.

In one embodiment of the invention, the first and second detectable labels comprise fluorescent labels.

In yet another embodiment of the invention a set of clones is described that is useful for diagnosing aneuploidies comprising the nucleic acid segments derived from cell lines with any from the group containing Turner syndrome, Klinefelter syndrome, XYY syndrome, or Triple X syndrome.

### BRIEF DESCRIPTION OF THE DRAWINGS

FIG. 1. Detection of triploidy and sex chromosome anomalies by SignatureChip® microarray analysis using a normal male (46,XY) as a control. Each clone is arranged along the x-axis according to its location on the chromosome with the most distal/telomeric p-arm clones on the left and the most distal/telomeric q-arm clones on the right. X chromosome clones begin on the left and Y chromosome clones are on the far right. The solid square ▪ line plots represent the ratios from the first slide for each patient (control Cy5/patient Cy3) and the open circle ∘ line plots represent the ratios obtained from the second slide for each patient in which the dyes have been reversed (patient Cy5/control Cy3). The expected X and Y chromosome ratios for each experiment are shown to the right of each plot. (A) Normal male (46,XY) vs. normal male (46,XY). (B) Normal female (46,XX) vs. normal male (46,XY). (C) 45,X vs. normal male (46,XY). (D) 47,XXY vs. normal male (46,XY). (E) Trisomy X (49,XXX,+2,+15) vs. normal male (46,XY). (F) 47,XYY vs. normal male (46,XY). (G) 48,XXYY vs. normal male (46,XY). (H) Triploid (70,XXY,+8) vs. normal male (46,XY). (I) Triploid (69,XXX) vs. normal male (46,XY).

FIG. 2. Detection of triploidy and sex chromosome anomalies by SignatureChip® microarray analysis using a Klinefelter male (47,XXY) as a control. The solid square ▪ line plots represent the ratios from the first slide for each patient (control Cy5/patient Cy3) and the open circle ∘ line plots represent the ratios obtained from the second slide for each patient in which the dyes have been reversed (patient Cy5/control Cy3). The expected X and Y chromosome ratios for each experiment are shown to the right of each plot. (A) Normal male (46,XY) vs. Klinefelter male (47,XXY). (B) Normal female (46,XX) vs. Klinefelter male (47,XXY). (C) 45,X vs. Klinefelter male (47,XXY). (D) 47,XXY vs. Klinefelter male (47,XXY). (E) Trisomy X (49,XXX,+2,+15) vs. Klinefelter male (47,XXY). (F) 47,XYY vs. Klinefelter male (47,XXY). (G) 48,XXYY vs. Klinefelter male (47,XXY). (H) Triploid (70,XXY,+8) vs. Klinefelter male (47,XXY). (I) Triploid (69,XXX) vs. Klinefelter male (47,XXY).

FIG. 3 SignatureChip® Profiles for artificially derived aneuploidies (right) and normal chromosome 1 (left). The solid square ▪ line plots represent the ratios from the first slide for experiment (Cy5:Cy3) and the open circle ∘ line plots represent the ratios obtained from the second slide for each experiment in which the dyes have been reversed (Cy3:Cy5).

### DETAILED DESCRIPTION

### Definitions

Unless defined otherwise, all technical and scientific terms used herein have the meaning commonly understood by a person skilled in the art to which this invention belongs. As used herein, the following terms have the meanings ascribed to them unless specified otherwise.

The terms “array” or “microarray” or “DNA array” or “nucleic acid array” as used herein is a plurality of target elements, each target element comprising a defined amount of one or more biological molecules, e.g., genomic nucleic acid segments, immobilized on a defined location on a substrate surface; as described in further detail, below.

The terms “cyanine 5” or “Cy5™” and “cyanine 3” or “Cy3™” refer to fluorescent cyanine dyes produced by Amersham Pharmacia Biotech (Piscataway, N.J.) (Amersham Life Sciences, Arlington Heights, Ill.), as described in detail, below, or equivalents. See U.S. Pat. Nos. 6,027,709; 5,714,386; 5,268,486; 5,151,507; 5,047,519. These dyes are typically incorporated into nucleic acids in the form of 5-amino-propargyl-2′-deoxy-cytidine 5′-triphosphate coupled to Cy5™ or Cy3™.

The terms “fluorescent dye” and “fluorescent label” as used herein includes all known fluors, including rhodamine dyes (e.g., tetramethylrhodamine, dibenzorhodamine, see, e.g., U.S. Pat. No. 6,051,719); fluorescein dyes; “BODIPY” dyes and equivalents (e.g., dipyrrometheneboron difluoride dyes, see, e.g., U.S. Pat. No. 5,274,113); derivatives of 1-[isoindolyl]methylene-isoindole (see, e.g., U.S. Pat. No. 5,433,896); and all equivalents. See also U.S. Pat. Nos. 6,028,190; 5,188,934.

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions. The term “stringent conditions” refers to conditions under which one nucleic acid will hybridize preferentially to second sequence (e.g., a sample genomic nucleic acid hybridizing to an immobilized nucleic acid probe in an array), and to a lesser extent to, or not at all to, other sequences. A “stringent hybridization” and “stringent hybridization wash conditions” in the context of nucleic acid hybridization (e.g., as in array, Southern or Northern hybridizations) are sequence dependent, and are different under different environmental parameters. Stringent hybridization conditions as used herein can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1M NaCl, and 1% SDS at 37° C., and a wash in 1×SSC at 45° C. Those of ordinary skill will readily recognize that alternative but comparable hybridization and wash conditions can be utilized to provide conditions of similar stringency.

However, the selection of a hybridization format is not critical, as is known in the art, it is the stringency of the wash conditions that set forth the conditions which determine whether a soluble, sample nucleic acid will specifically hybridize to an immobilized nucleic acid. Wash conditions can include, e.g.: a salt concentration of about 0.02 molar at pH 7 and a temperature of at least about 50° C. or about 55° C. to about 60° C.; or, a salt concentration of about 0.15 M NaCl and a temperature of at least about 72° C. for at least about 15 minutes; or, a salt concentration of about 0.2×SSC at a temperature of at least about 50° C. or about 55° C. to about 60° C. for at least about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C. See Sambrook, Ausubel, or Tijssen (cited herein) for detailed descriptions of equivalent hybridization and wash conditions and for reagents and buffers, e.g., SSC buffers and equivalent reagents and conditions.

The phrase “labeled with a detectable composition” or “labeled with a detectable moiety” as used herein refers to a nucleic acid comprising a detectable composition, i.e., a label, as described in detail, below. The label can also be another biological molecule, as a nucleic acid, e.g., a nucleic acid in the form of a stem-loop structure as a “molecular beacon,” as described below. This includes incorporation of labeled bases (or, bases which can bind to a detectable label) into the nucleic acid by, e.g., nick translation, random primer extension, amplification with degenerate primers, and the like. The label can be detectable by any means, e.g., visual, spectroscopic, photochemical, biochemical, immunochemical, physical or chemical means. Examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin.

The term “nucleic acid” as used herein refers to a deoxyribonucleotide or ribonucleotide in either single- or double-stranded form. The term encompasses nucleic acids containing known analogues of natural nucleotides. The term also encompasses nucleic-acid-like structures with synthetic backbones. DNA backbone analogues provided by the invention include phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, and peptide nucleic acids (PNAs); see Oligonucleotides and Analogues, a Practical Approach, edited by F. Eckstein, IRL Press at Oxford University Press (1991); Antisense Strategies, Annals of the New York Academy of Sciences, Volume 600, Eds. Baserga and Denhardt (NYAS 1992); Milligan (1993) J. Med. Chem. 36:1923-1937; Antisense Research and Applications (1993, CRC Press). PNAs contain non-ionic backbones, such as N-(2-aminoethyl) glycine units. Phosphorothioate linkages are described, e.g., by U.S. Pat. Nos. 6,031,092; 6,001,982; 5,684,148; see also, WO 97/03211; WO 96/39154; Mata (1997) Toxicol. Appl. Pharmacol., 144:189-197. Other synthetic backbones encompassed by the term include methyl-phosphonate linkages or alternating methylphosphonate and phosphodiester linkages (see, e.g., U.S. Pat. No. 5,962,674; Strauss-Soukup (1997) Biochemistry 36:8692-8698), and benzylphosphonate linkages (see, e.g., U.S. Pat. No. 5,532,226; Samstag (1996) Antisense Nucleic Acid Drug Dev 6:153-156). The term nucleic acid is used interchangeably with gene, DNA, RNA, cDNA, mRNA, oligonucleotide primer, probe and amplification product.

The term “genomic DNA” or “genomic nucleic acid” includes nucleic acid isolated from a nucleus of one or more cells, and, includes nucleic acid derived from (e.g., isolated from, amplified from, cloned from, synthetic versions of) genomic DNA. The genomic DNA can be from any source, as discussed in detail, below. The term “wild type genomic nucleic acid” means a sample of genomic nucleic acid having no known or substantially no known contiguous gene abnormalities.

The term “a sample comprising a nucleic acid” or “sample of nucleic acid” as used herein refers to a sample comprising a DNA or an RNA, or nucleic acid representative of DNA or RNA isolated from a natural source, in a form suitable for hybridization (e.g., as a soluble aqueous solution) to another nucleic acid or polypeptide or combination thereof (e.g., immobilized probes). The nucleic acid may be isolated, cloned or amplified; it may be, e.g., genomic DNA, episomal DNA, mitochondrial DNA, mRNA, or cDNA; it may be a genomic segment that includes, e.g., particular promoters, enhancers, coding sequences, and the like; it may also include restriction fragments, cDNA libraries or fragments thereof, etc. The nucleic acid sample may be extracted from particular cells, tissues or body fluids, or, can be from cell cultures, including cell lines, or from preserved tissue sample, as described in detail, below.

The term aneuploidy as described here refers to an aberration in which the chromosome number of an individual is different from the normal set for the species.

The term copy number refers to the number of copies of any gene or random DNA segment in a given cell.

### General Techniques

Novel methods and systems for simultaneous testing of numerous genetic alterations that occur in different parts of the human genome are shown in application Ser. No. 11/057,088 published Feb. 11, 2005, the entire contents of which are incorporated herein by reference. The methods, arrays and controls of this invention are used for detecting aneuploidy and other unbalanced chromosomal rearrangements in biological samples, (e.g., products of conception (POCs)).

The subject matter described herein can also be used in various fields to greatly improve the accuracy and reliability of nucleic acid analyses, chromosome mapping, and genetic testing, e.g., for diagnosing an unbalanced distribution of chromosomes or chromosomal segments and assessing the quality of CGH array experiments. Novel control nucleic acids are described which can generate a standard curve of ratios in a CGH array assay. The novel control nucleic acids can be non-complementary to nucleic acid in the test sample and hybridize to target elements on the array which do not bind test sample. The ratio of added artificial or naturally occurring imbalanced nucleic acid added to the test and/or reference sample provide a reliable reference for determining the quality of the array experiment with respect to labeling and hybridization efficiency. Alternatively, control nucleic acids can be used that are similar to chromosomal loci in the test sample but that are not part of the chromosomal region being tested.

Selected chromosomal target elements can be included on the array which are only complementary to control nucleic acid and do not hybridize to nucleic acid in the test sample. Selected nucleic acids are described for use as control target elements on an array. Selected nucleic acids are described for use as reference control samples in an exemplary method.

### Array Target Elements

In an implementation that uses a diagnostic array (hereafter, “array”), such as a microarray used for comparative genomic hybridization (CGH), a comprehensive battery of clinically relevant chromosomal loci are carefully selected and screened to provide stringent diagnostic efficacy.

The invention describes a microarray useful for the diagnosis of aneuploidy. In one embodiment of the invention microarray contains contigs of three or more BAC clones across the human genome, including more than 10, 20, 30 or 40 subtelomeric regions, and more than 10, 20, 30 or 40 pericentromeric regions, and more than 10, 20, 30 or 40 known microdeletion syndromes. In one embodiment of the invention the microarray comprises elements complementary to pseudoautosomal loci. Pseudoautosomal loci are located on both the X and Y chromosome respectively. The signal generated from test sample hybridization to these elements on a microarray is a measure of the total quantity of sex chromosome present. Thus, the signal from pseudoautosomal elements provides for an enhanced detection of sex chromosome aneuploidy.

The invention describes a microarray wherein the overall genetic code sequence (“base-pair sequence”) of each clinically relevant chromosomal locus selected for an array may be parsed or disassembled into multiple contiguous and/or overlapping segments. Each segment-as represented by a nucleic acid (e.g., a clone or synthesized oligonucleotide) is isolated into a reliable individual test area (“target”) on an exemplary array, free from interfering influences (on hybridization) of clones that represent adjacent sequences that occur on a native chromosome. A nucleic acid representing an individual segment of the base-pair sequence of a chromosomal locus may be included multiple times and in different positions within different sub-arrays of an exemplary array.

When a patient's chromosomal material is tested against the array of this invention, redundant occurrences of a given individual segment test are compared with each other, and test results of the multiple segments of a locus are collated, that is, logically reassembled back into a single combined test result for the overall base-pair sequence of the locus. In one implementation, overlapping segments can also be used for increased test resolution and certainty of diagnosis.

In one implementation of an array of this invention, copies of clones assigned to a segment of a chromosomal locus may be placed into target elements in multiple sub-arrays to provide reliability and comparison. Moreover, groups of sub-arrays may be redundantly repeated in an exemplary array. On a larger scale, an array consisting of redundantly repeated sub-arrays may itself be redundantly repeated so that the arrangement motif of the smallest sub-arrays is redundantly repeated on multiple larger scales.

One or more control target elements of this invention can be added to the array to specifically recognize nucleic acid added to either the reference or test sample or both. Control targets typically remain unaltered despite alteration in a diagnostic target of interest. Another embodiment of control target elements of this invention is the use of multiple regions of the genome (e.g., 3-5 regions of the genome for each desired control ratio) so that the likelihood of having a test sample with copy number differences at more than one of these locations would be very small. Control target elements can be derived from the test sample species (e.g., human) or from a species different from the test sample (e.g., Drosophila, yeast, E. coli). Nucleic acid that is added in known quantities to either the reference sample, test sample or both will hybridize to the control target elements and generate a signal that can be compared to the signal from the test sample in order to determine the quality of the array experiment with respect to labeling and hybridization efficiency.

One aspect of the current invention is that the ratio of signal from control target elements generated by added nucleic acid that has been added to both the test sample and reference sample provides a reliable reference for the quantization and analysis of CGH array assays.

An exemplary array implementation provides a comprehensive battery of diverse tests that can be performed relatively inexpensively compared to costly separate conventional tests. Moreover, such an exemplary array provides unprecedented accuracy, reliability, and convenience for diagnosing a myriad of genetic alterations, for example, in one single CGH microarray test.

### Control Nucleic Acid

In array based comparative genomic hybridization (array CGH) genomic nucleic acid from a test sample and a reference control sample are oppositely labeled and co-hybridized to an array of nucleic acid targets elements. The reference control nucleic acid is generally genomic nucleic acid from a phenotypiclly normal male or female individual or pool of individuals. One approach to the analysis of CGH array experiments is to co-hybridize labeled genomic nucleic acid from a test sample to oppositely labeled genomic nucleic acid from a normal reference control of the opposite sex. This approach creates a built-in positive control for the entire array CGH experiment due to the inherent differences in copy number of the sex chromosomes between chromosomally normal males (46,XY) and chromosomally normal females (46,XX).

Using a reference nucleic acid of the opposite sex in array CGH experiments is important in array CGH because it verifies that the experiment has been performed correctly and that the assay is capable of yielding the appropriate results, but can make it difficult to distinguish other subtle sex chromosome anomalies due to the fact that the sex chromosomes will already show a 1:2 X chromosome copy number difference and a 0:1 Y chromosome copy number difference between the test sample and the control (male:female). This is particularly crucial for diagnostic array CGH experiments which are performed with the intent of yielding a definitive diagnostic result regarding the relative copy number of the regions of the patient's genome being interrogated by the array.

An embodiment of this invention provides for the use of nucleic acid segments from cells which comprise naturally occurring or artificially created polyploidies as controls to more accurately identify chromosomal imbalance in a test sample. The controls of this invention overcome difficulties encountered by both conventional and array CGH when dealing with ploidies which stem from the fact that CGH detects dosage differences by comparing two equal quantities of genomic DNA extracted from two cell lines with potentially different genomic contents. In the case of triploids, the quantity of DNA present in a given number of triploid cells is greater than the quantity of DNA present in an equivalent number of diploid cells. Thus, when a triploid test sample is compared to a diploid reference sample by array CGH, obtaining the true dosage difference ratio of 3:2 (triploid:diploid) for all chromosomes would require comparing a given quantity of DNA from the diploid sample (e.g., 500 ng) to a 50% larger quantity of DNA from the triploid sample (e.g., 750 ng). Therefore, when equal quantities of extracted diploid and triploid genomic DNAs are compared, chromosome copy numbers will actually be underrepresented in the triploid sample by one third. For this reason, the extra haploid set of chromosomes in a triploid test sample will be missed, when compared to a diploid control, because the relative copy number ratio of each chromosome will appear to be equivalent. In other words, when comparing equal quantities of genomic DNA, a triploid test sample will appear to have a copy number equivalent to 2 (0.67×3) for each chromosome and the diploid control sample will have the normal copy number of 2 which yields a triploid test:control sample ratio of essentially 2:2. This would be equivalent to a diploid test sample compared to diploid control sample. The major advantage of a using a control sample with a known aneuploidy as a reference over a chromosomally balanced reference is that it makes it easier to distinguish chromosomal abnormalities from normal chromosome complements by identifying single copy number changes of the sex chromosomes and the pseudoautosomal regions.

Although using a 46,XY reference can identify sex chromosome copy number changes, in practice, any variability in the quality of the hybridization may make it difficult to distinguish between the sex chromosome copy number gains because the log₂ values are only slightly higher as the ratios increase. For example, when using a 46,XY reference, a 46,XX test sample has an X chromosome ratio of 2:1 (test sample:reference) and a theoretical log₂ value of 1, a 47,XXX test sample has an X chromosome ratio of 3:1 and a log₂ value of 1.58, and a 48,XXXX test sample has an X chromosome ratio of 4:1 and a log₂ value of 2. In practice, these subtle changes in the log₂ values can be difficult to distinguish because the observed values rarely reach their theoretical values. Thus, using a 46,XY reference forces the analysis software (and diagnostician) to distinguish between sometimes subtle 2:1 (46,XX) and 3:1 (47,XXX) copy number changes of the X chromosome for all normal 46,XX cases.

When an embodiment of this invention, 47,XXY reference sample is used as a control, a normal 46,XY has an X chromosome ratio of 1:2 (test sample:reference sample) with a log₂ value of −1 indicating a single copy loss, a normal 46,XX has an equal X chromosome ratio of 2:2 with a log₂ value of 0, and a 47,XXX has a single copy X chromosome gain with a ratio of 3:2 and a log₂ value of 0.58. Because most patient samples tested in a clinical setting are likely to have normal copy numbers of the X and Y chromosomes, using a 47,XXY reference reduces the majority of the analyses performed to the more easily distinguishable single copy X chromosome losses (46,XY) and no copy number change (46,XX). In addition, the more rare 47,XXX case will be easily distinguished from a normal 46,XX by a single copy gain of the X chromosome.

Another advantage of using an unbalanced chromosomal sample such as 47,XXY as a reference sample is that it allows for a simple distinction between the even more rare 47,XXX and 48,XXXX cases by identifying single copy gains of the pseudoautosomal regions. For example, when using a 46,XY reference sample, a 47,XXX test sample has a pseudoautosomal region ratio of 3:2 with a log₂ value of 0.58 and a 48,XXXX test sample has a pseudoautosomal region ratio of 4:2 with a log₂ value of 1. In practice, the difference between these two values may be subtle, making it difficult to distinguish between these two ratios. However, when a 47,XXY reference is used, a 47,XXX has a pseudoautosomal region ratio of 3:3 and a log₂ value of 0, whereas a 48,XXXX will have a pseudoautosomal region ratio of 4:3 and a log₂ value of 0.42 indicating a single copy number gain.

Table 2 lists the theoretical ratios expected to be observed in array CGH experiments for the sex chromosomes when test samples with sex chromosome aneuploidies and polyploidies are compared to either a normal diploid male (46,XY) or a Klinefeleter male (47,XXY) as the reference control. Using DNA from a Klinefelter cell line as a reference control nucleic acid in array CGH produces clearer results when analyzing some sex chromosome anomalies such as trisomies and tetrasomies of the X chromosome by reducing the analysis to essentially single copy gains (Table 2). Furthermore, careful attention to the observed sex chromosome ratios (subject/control) in array CGH experiments can allow for the discrimination of a variety of sex chromosome imbalances as well as some common triploidies (69,XXY and 69,XYY). In the cases of 69,XXY and 69,XYY triploidies, careful attention to the sex chromosome ratios can distinguish these triploidies from normal cell lines because, unlike the 69,XXX triploidy, the X and Y chromosomes are not present in three copies per cell. However, all of the autosomes will be present in three copies per cell and will appear to have the normal 2:2 ratios for the reasons described above.

The method of this invention utilizes control reference nucleic acid with abnormal amounts of one or more chromosomes relative to the majority of chromosomes in the control sample. In this way, the ratios of the chromosomes in the control reference nucleic acid provide a benchmark of the range of chromosomal intensities for known quantities of nucleic acid. The intensity of test results can be interpreted with greater ease and triploid detection is achieved with greater confidence. Control nucleic acid can be derived from cell lines with known chromosomal imbalance or can be artificially created with imbalanced copy numbers by the addition of extraneous chromosomal material from one or more chromosomes.

Artificial aneuploidies of this invention are generated by the addition of nucleic acid segments to test or reference samples. Artificial aneuploidies of this invention provide positive controls for other desirable ratios such as 3:2, 3:1, 3:4, 2:0, etc. Furthermore, positive controls for the opposite ratios of any of these ratios would also be advantageous (e.g., 2:3, 1:3, 4:3, 0:2, 2:1, 1:0, etc.) in diagnosing sample results and confirming that an experiment is performing correctly. Conventional non sex-matched controls do not provide a range of ratios for comparison. Therefore, an additional nucleic acid added to the reference, test or both samples provides an alternative internal positive control for array CGH array assays which provides for a more accurate analysis of the assay results.

In one embodiment of the invention a plurality of nucleic segments added in various amounts to test and control samples is used to generate a standard curve of ratios. The signal from the standard curve in a hybridization experiment provides a useful measure of the degree of signal change relative to the amount of each chromosome in the test sample.

One embodiment of the present invention comprises using added nucleic acid segments as an internal positive control to create an artificial aneuploidy condition in the test sample, reference sample or both. This would allow for the desired experimental controls to be selected by the diagnostician and could potentially be unlimited in the ratios desired to be controlled for. This would also allow for the use of sex matched controls because the artificial aneuploidy would provide the positive control for the experiment. In one embodiment of the invention, human nucleic acid sequences from bacterial artificial chromosomes (BACs) or any other vectors containing human DNA inserts (BACs, PACs, cosmids, fosmids, etc) could be added to the test sample and/or reference sample such that the desired ratio of those particular sequences are obtained when array CGH is performed.

In another embodiment of the invention, a plurality of human nucleic acid sequences from flow sorted human chromosomes could be added to the test sample and/or reference sample such that the desired ratio of those particular sequences is obtained when array CGH is performed. In one embodiment of this invention, the desired ratios of a plurality of added nucleic acid segments generate a standard curve of ratios when hybridized such that patient chromosome number can be more accurately measured by CGH array analysis. When the artificial aneuploidy comprises human nucleic acid segments, care must be taken to insure that the potential abnormality in the patient is not masked. This is achieved in one embodiment by choosing multiple regions of the genome that correspond to G-positive bands of the chromosomes since these regions contain few genes and are therefore less likely to have a copy number abnormality that is responsible for a patient's phenotype. In another embodiment of this invention multiple regions of the genome (say 3-5 regions of the genome for each desired artificial aneuploidy) are used so that the likelihood of having a patient with copy number differences at more than one of these locations would be very small.

In another embodiment of this invention, artificial aneuploidies can be generated by non-human nucleic acid sequences (e.g., yeast, Drosophila, E. coli). These are added to the test sample and/or reference sample such that the desired ratio of those particular sequences is obtained when array CGH is performed. Non-human nucleic acid sequences could be from the whole genome or from specific genomic regions that have been isolated from any one of a variety of vectors containing DNA inserts or can be synthetically generated. In the embodiments of the invention where non-human nucleic acid is added to either the reference or test sample or both, it is necessary to have the appropriate target sequences spotted on the microarray. In one embodiment of the invention, a plurality of non-human nucleic acid segments from a plurality of genomic regions are added to test and/or reference sample such that the ratios of the of segments generate a standard curve which aids in the interpretation of hybridization results.

Artificial aneuploidies provide additional features for a CGH array assay. In one embodiment of the invention hybridization of nucleic acid that comprises an artificial aneuploidy provides for quality control of the microarray by confirming the location of individual elements after the production of one or more microarrays. One non-limiting example of this application comprises adding an additional copy of all of the BAC clones corresponding to chromosome 22 that are represented on a microarray to a test or reference sample. This would result in a single copy gain of all those clones in the corresponding sample. Furthermore, all of the chromosome 22 clones could be identified on the array and it could be determined whether or not they are in the correct location and if they are working correctly in the array CGH assay. This would be very useful for microarray production purposes in that each lot of microarrays could be tested to confirm that each spot is in the correct location and functioning correctly.

Another application for artificial aneuploidies is to provide a unique identifier for each patient as it passes through the laboratory. This is a quality control procedure that would ensure that the sample of blood from which a patient's DNA is extracted is identified at the end of the array CGH assay by the unique artificial aneuploidy that is assigned to that individual sample. In one non-limiting example, a sample's genomic DNA is spiked with known volumes of 5-10 different nucleic acid segments so as to produce additional signal in that sample for those the corresponding target elements. When the array CGH analysis is complete for that sample, the additional signal that is observed in the final analysis should correspond to what was added in the original genomic DNA (or sample specimen). This ensures that there were no sample mix-ups at any of the intermediate steps of the array CGH procedure. The additional signal could be generated with a variety of human or nonhuman nucleic acid segments as long as there are corresponding targets on the microarray.

### Hybridization Conditions

In general, both the test and control nucleic acids are labeled with each of detectable label (e.g., Cy3™ and Cy5™), and two mixtures are made for a “dye swap” analysis. Many commercial instruments are designed to accommodate the detection of these two dyes. To increase the stability of Cy5™, or fluors or other oxidation-sensitive compounds, antioxidants and free radical scavengers can be used in hybridization mixes, the hybridization and/or the wash solutions. Thus, Cy5™ signals are dramatically increased and longer hybridization times are possible.

In practicing the methods of the invention, samples of nucleic acid, e.g., isolated, cloned or amplified genomic nucleic acid, are hybridized to the compilations, or sets, libraries or collections of the invention or arrays of the invention, including immobilized nucleic acids. In alternative aspects, the hybridization and/or wash conditions are carried out under moderate to stringent conditions.

An extensive guide to the hybridization of nucleic acids is found in, e.g., Sambrook Ausubel, Tijssen. Stringent hybridization and wash conditions can be selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe.

In alternative aspects, the methods of the invention are carried out in a controlled, unsaturated humidity environment, and, the compilations, or sets, libraries or collections, of nucleic acids or arrays of the invention can further comprise apparatus or devices capable of controlling humidity. Controlling humidity is one parameter that can be manipulated to increase hybridization sensitivity. Thus, in one aspect, in practicing the methods of the invention, hybridization can be carried out in a controlled, unsaturated humidity environment; hybridization efficiency is significantly improved if the humidity is not saturated. The hybridization efficiency can be improved if the humidity is dynamically controlled, i.e., if the humidity changes during hybridization. Array devices comprising housings and controls that allow the operator to control the humidity during pre-hybridization, hybridization, wash and/or detection stages can be used. The device can have detection, control and memory components to allow pre-programming of the humidity (and temperature and other parameters) during the entire procedural cycle, including pre-hybridization, hybridization, wash and detection steps.

In alternative aspects, the methods of the invention can incorporate hybridization conditions comprising temperature fluctuations and, the compilations, or sets, libraries or collections, of nucleic acids or arrays of the invention can further comprise apparatus or devices capable of controlling temperature, e.g., an oven. Hybridization has much better efficiency in a changing temperature environment as compared to conditions where the temperature is set precisely or at relatively constant level (e.g., plus or minus a couple of degrees, as with most commercial ovens). Reaction chamber temperatures can be fluctuatingly modified by, e.g., an oven, or other device capable of creating changing temperatures.

In alternative aspects, the methods of the invention can comprise hybridization conditions comprising osmotic fluctuations, and, the compilations, or sets, libraries or collections, of nucleic acids or arrays of the invention can further comprise apparatus or devices capable of controlling osmotic conditions, e.g., generate a solute gradient. Hybridization efficiency (i.e., time to equilibrium) can also be enhanced by a hybridization environment that comprises changing hyper-/hypo-tonicity, e.g., a solute gradient. A solute gradient is created in a device. For example, a low salt hybridization solution is placed on one side of the array hybridization chamber and a higher salt buffer is placed on the other side to generate a solute gradient in the chamber.

### High Availability Aspect

High availability (HA) is a term used between artisans in various trades to refer to a system that is capable of providing reliable and/or accurate service almost all of the time. Provision of reliable HA genetic testing has been a longstanding problem. In one aspect of the subject matter, the exemplary methods described herein achieve HA genetic testing by creating very reliable components, by creating elements that are fault tolerant, by creating relationships between the elements that avoid error, and by creating subsystems that are backed up by redundant provisioning. In an exemplary array, “redundant provisioning” and “clustering” are design techniques that impart HA to genetic testing. Nucleic acids used as reference controls are selected to provide quality information and diagnostic efficacy. The use of naturally occurring and artificially created abnormally balance nucleic acids as reference control on CGH arrays strategically eliminates many possibilities for hybridization errors and errors introduced by variations that occur within usual procedural tolerances.

### EXAMPLE

### Example 1

In a blinded study, 19 biological samples derived from either products of conception (POCs) with abnormal karyotypes or cell lines with chromosomal abnormalities common to POCs were tested with arrays and control DNA of this invention These biological samples included seven trisomies, two multiple aneuploidies, seven sex chromosome aneuploidies including two 45,X, and three triploidies.

The 19 transformed cell lines derived from POCs or cell lines with chromosomal aberrations commonly found in POCs were obtained from Coriell (Camden, N.J.) and the cytogenetics laboratory at Sacred Heart Medical Center (Spokane, Wash.), were de-identified. The analysis on eight of these subjects was previously reported as part of the development and validation of the SignatureChip® microarray (Bejjani et al. 2005) (Table 1). Control DNAs used in this study were extracted from peripheral blood of a chromosomally normal male and a chromosomally normal female individual and from a Klinefelter male cell line (47,XXY) (Coriell).

Array CGH was performed on 11 cell lines and two normal individuals (one male and one female) using the SignatureChip® microarray (Signature Genomic Laboratories, LLC, Spokane, Wash.) as previously described in U.S. patent application Ser. No. 11/057,088 (Shaffer et al., 2005). Briefly, genomic DNAs were extracted using a Puregene DNA isolation kit (Gentra Systems, Inc. Minneapolis, Minn.), digested with Dpn II (New England Biolabs, Inc., Beverly, Mass.), and reprecipitated prior to labeling. A dye-reversal strategy was used on two separate microarrays in which 500 ng of both test subject and reference control DNAs were labeled (Bio Prime DNA labeling System, Invitrogen, Carlsbad, Calif.) with Cy3™ or Cy5™, respectively (Hodgson et al. 2001). Test subject and reference control DNAs were co-hybridized to one microarray and then oppositely labeled and co-hybridized to a second microarray (Wessendorf et al. 2002). Prior to hybridization, slides were blocked using 10% bovine serum albumin fraction V (Sigma, St. Louis, Mo.) and 20 μg salmon sperm DNA (Invitrogen, Carlsbad, Calif.).

Images of the hybridized slides were acquired using a GenePix 4000B dual-laser scanner (Axon Instruments, Union City, Calif.) by simultaneously scanning each array at wavelengths of 635 nm and 532 nm. Spots were analyzed with GenePix Pro 5.0 imaging software (Axon Instruments, Union City, Calif.). The mean ratio of fluorescence intensities derived from hybridized subject and reference control DNA at each test spot on the microarray was calculated and normalized by the mean ratios measured from reference spots on the same slide.. Because each clone is printed in quadruplicate on the SignatureChip™ microarray, the mean ratio of the four normalized spots for each clone was obtained, subsequently converted to a log₂ scale, and plotted in Microsoft Excel™.

All samples were hybridized to two microarrays using a dye-reversal strategy. All abnormal gains (trisomies) and losses (45, X) were correctly identified. Detecting the triploidies and the X chromosome gains in trisomy X cases required careful interpretation of the microarray results with particular attention to the sex chromosome ratios between the patient sample and the control.

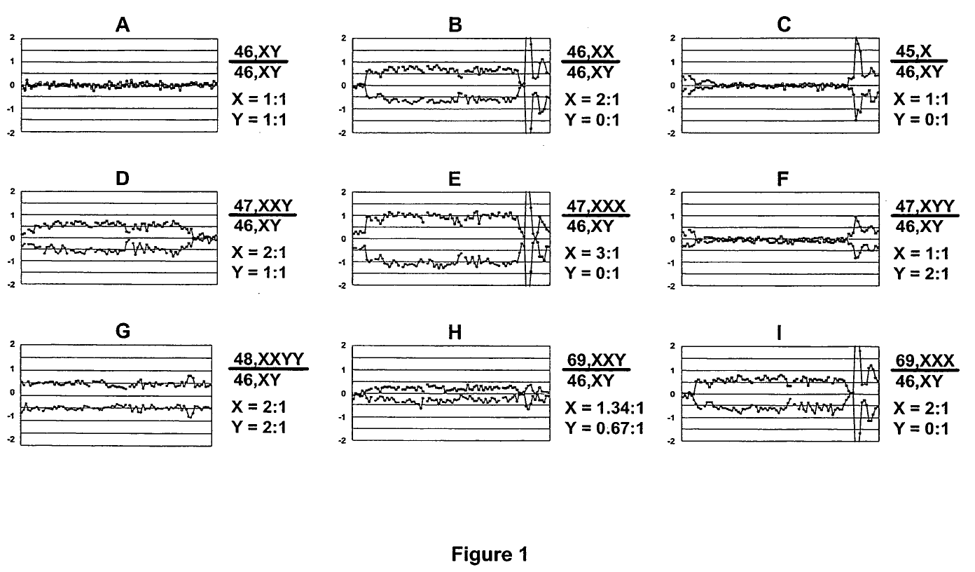

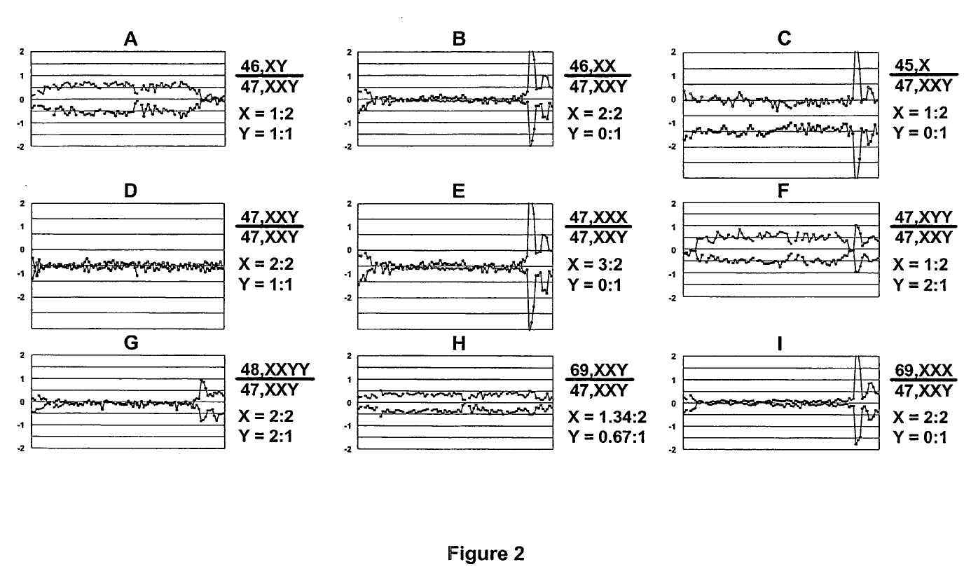

The array CGH results on all 19 cases are shown Table 1. Excluding the triploid cases, were not correctly when using a normal male (46, XY) as a reference control (FIG. 1). However, when using a normal male (46, XY) as a reference control, the distinction between a normal 46,XX test subject and a test subject with trisomy X (47,XXX) was subtle with only slightly higher ratio values across the X chromosome for the trisomy X than for the normal diploid female (FIG. 1B and E). Therefore, we repeated the hybridizations on all 11 subjects with sex chromosome aneuploidies and triploidies using a Klinefelter male cell line (47,XXY) as a reference control (FIG. 2). In these experiments, the trisomy X was clearly distinguishable from a normal diploid female (46,XX) due to the fact that the ratios of the sex chromosomes had been reduced to the identification of single copy gains and losses (FIG. 2B and E). In addition, analysis of the triploid cell lines (69,XXY) using either a normal male or a Klinefelter male cell line as the reference control produced characteristic plots that were distinguishable from normal diploid cell lines.

TABLE 1
Results of array CGH analysis using the SignatureChip ™ microarray
on 19 POCs and cell lines with known chromosome abnormalities.

| | |
|---|---|
| Array CGH Results | Concordance |

| Sample | Known | | chromosome | between array |
|---|---|---|---|---|
| Number | Karyotype | gain/loss | or region | and karyotype |
| | Trisomies | | | |
| 04-000076^{a} | 47, XX, +9 | Gain | 9 | Yes |
| 04-000082^{a} | 47, XY, +15 | Gain | 15 | Yes |
| 04-000083^{a} | 47, XX, +16 | Gain | 16 | Yes |
| 04-000077^{a} | 47, XX, +18 | Gain | 18 | Yes |
| 04-000078^{a} | 47, XX, +21 | Gain | 21 | Yes |
| 04-000079^{a} | 47, XY, +21 | Gain | 21 | Yes |
| 04-000081^{a} | 47, XY, +21 | Gain | 21 | Yes |
| 04-000074^{a} | 48, XY, +2, | Gain | 2, 13 | Yes |
| | +13 | | | |
| | Sex | | | |
| | Chromosome | | | |
| | Aneuploidies | | | |
| 04-000067 | 45, X | Loss | X | Yes |
| 04-000110 | 45, X | Loss | X | Yes |
| SHMC 001 | 47, XXX | Gain | X | Yes |
| GM03102 | 47, XXY | Gain | X | Yes |
| GM00325 | 47, XXY | Gain | X | Yes |
| GM11337 | 47, XYY | Gain | Y | Yes |
| GM04375 | 48, XXYY | Gain | X, Y | Yes |
| 04-000092 | 49, XXX, | Gain | 2, 15, X | Yes |
| | +2, +15 | | | |
| | Triploidies | | | |
| SHMC 003 | 69, XXX | None | | No^{b} |
| SHMC 002 | 69, XXY | Gain | triploidy | Yes |
| 04-000091 | 70, XXY, +8 | Gain | 8, triploidy | Yes |

^{a}Chromosome abnormalities in these subjects were previously reported (Bejjani et at. 2005).
^{b}69, XXX cell lines are undetectable by array CGH. As expected, the array CGH analysis for the 69, XXX cell line was indistinguishable from results obtained for a normal 46, XX female sample (see text for details).

TABLE 2
Theoretical sex chromosome ratios (test sample:reference
control) expected in CGH experiments when test samples with normal
karyotypes, sex chromosome aneuploidies, triploidies, or tetraploidies are
compared to equal quantities of genomic DNA from either a normal male or
Klinefelter cell line.

| | | | | | Analysis |
|---|---|---|---|---|---|
| | Reference Control | | | | facilitated |
| | 46, XY | | 47, XXY | | by use of a |
| | X | Y | X | Y | Klinefelter |
| Test Sample | Ratio | Ratio | Ratio | Ratio | Control |

Normal

| | | | | | |
|---|---|---|---|---|---|
| 46, XY^{a} | 1:1 | 1:1 | 1:2 | 1:1 | No |
| 46, XX^{b} | 2:1 | 0:1 | 2:2 = 1:1 | 0:1 | No |

Sex Chromosome Aneuploidy

| | | | | | |
|---|---|---|---|---|---|
| 45, X | 1:1 | 0:1 | 1:2 | 0:1 | No |
| 47, XXY | 2:1 | 1:1 | 2:2 = 1:1 | 1:1 | No |
| 47, XXX | 3:1 | 0:1 | 3:2 = 1.5:1 | 0:1 | Yes^{c} |
| 47, XYY | 1:1 | 2:1 | 1:2 | 2:1 | No |
| 48, XXYY | 2:1 | 2:1 | 2:2 = 1:1 | 2:1 | No |
| 48, XXXX | 4:1 | 0:1 | 4:2 = 2:1 | 0:1 | Yes^{c} |
| 49, XXXXY | 4:1 | 1:1 | 4:2 = 2:1 | 1:1 | Yes^{c} |

Triploidy^{d}

| | | | | | |
|---|---|---|---|---|---|
| 69, XXX^{b} | 2:1 | 0:1 | 2:2 = 1:1 | 0:1 | No |
| 69, XXY | 1.34:1 | 0.67:1 | 1.34:2 = 0.67:1 | 0.67:1 | No |
| 69, XYY | 0.67:1 | 1.34:1 | 0.67:2 = 0.34:1 | 1.34:1 | No |

Tetraploidy^{e}

| | | | | | |
|---|---|---|---|---|---|
| 92, XXYY^{a} | 1:1 | 1:1 | 1:2 | 1:1 | No |
| 92, XXXX^{b} | 2:1 | 0:1 | 2:2 = 1:1 | 0:1 | No |

^{a}Sex chromosome ratios for test samples with 46, XY or 92, XXYY karyotypes are indistinguishable from each other regardless of the reference control used.
^{b}Sex chromosome ratios for test samples with 46, XX, 69, XXX, or 92, XXXX karyotypes are indistinguishable from each other regardless of the reference control used.
^{c}The use of a Klinefelter male (47, XXY) cell line as a control can facilitate the detection of some sex chromosome aneuploidies by reducing the X chromosome ratios to single copy differences.

### Example 2

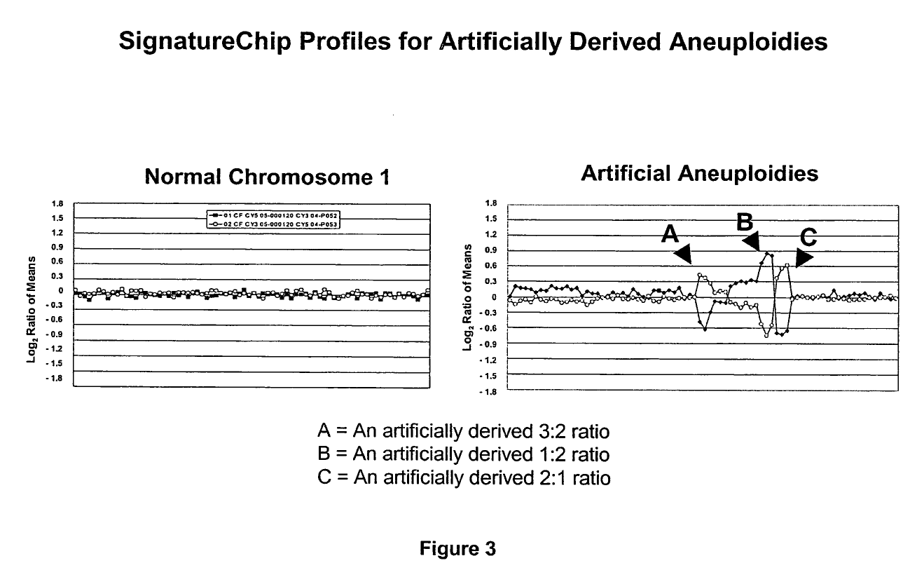

An artificial aneuploidy is created by adding additional nucleic acid to a test and control sample. This is used to improve the accuracy of the analysis of a test sample. Human BAC DNAs from three overlapping clones that are currently represented on the SignatureChip™ microarray are pooled and diluted. The DNAs are added to the test and reference sample in the amounts indicated in Table 3. Both the test and the reference samples each have two copies of the genes before the BAC clones are added. For example, adding 1 volume to the test sample and 2 volumes to the reference control will produce a 3:4 final ratio (test:control) in the array CGH experiment because there are already 2 copies of each sequence (from each of the two chromosomes) in the genomic DNA of the test DNA and 2 copies in the genomic DNA of the reference control. The final ratios produced are 3:2 (by adding only 1 volume to the test sample), 1:2 (by adding 4 volumes to the patient and 1 to the control), and 2:1 (by adding only 2 volumes to the test sample). FIG. 3 shows the change in signal caused by the additional DNA in the sample.

TABLE 3
Volumes of added chromosome 1 to test and reference
samples.

| | Test sample | Reference sample | |
|---|---|---|---|
| Added DNA | (Volume) | (Volume) | Expected ratio^{a} |
| Hybridization A | 1 | 0 | 3:2 |
| Hybridization B | 4 | 1 | 1:2 |
| Hybridization C | 2 | 0 | 2:1 |

^{a}Because the test and reference samples are normal diploid samples, they already have two copies of the region of the genome for which an artificial aneuploid is being generated. Thus, the expected ratios in the table reflect the addition of the specified volumes to the two existing “volumes” or quantities of nucleic acid for that particular region of the genome.

The signal from standard curves generated by nucleic acid added to experimental samples enables CGH experiments to be evaluated with greater accuracy. In addition, the quality of hybridizations can be determined for each experiment.

A number of embodiments of the invention have been described. Nevertheless, it will be understood that various modifications may be made without departing from the spirit and scope of the invention. Accordingly, other embodiments are within the scope of the following claims.

What is claimed is:

1. An array for the detection of aneuploidy that comprises a first plurality of nucleic acid segments corresponding to a first set of chromosomal loci wherein each chromosomal locus is capable of genetic alteration indicative of a copy number and a second plurality of nucleic acid segments corresponding to a second set of chromosomal loci that are not associated with the copy number being detected, wherein each of the first and second plurality of nucleic acid segments represents a portion of the base-pair sequence of a chromosomal locus; wherein each nucleic acid segment is immobilized to a discrete and known spot on a substrate surface to form an array of nucleic acids, wherein the nucleic acid segments representing chromosomal loci that are adjacent on a native chromosome are placed in non-adjacent target areas of the array.

2. The array of claim 1 wherein the aneuploidy comprises sex chromosomes imbalances.

3. The array of claim 1 wherein the type of aneuploidy is selected from the group consisting of Turner syndrome (45,X), Klinefelter syndrome 47,XXY, 47,XXX, 45,X/46,XY, 46,XX/(male), 46,XX/46,XY(female), 47,XYY, 46,XY/47,XYY, 45,X/46,XY (male), 46,XX/47,XX,del(Yp) (female), or 46,XX/46,XY(female).

4. The array of claim 1 wherein the aneuploidy comprises non-sex chromosome imbalances.

5. The array of claim 1, wherein the second plurality of nucleic acids is selected from the group consisting of telomeric, pericentromeric, and pseudoautosomal chromosomal loci.

6. The array of claim 1 wherein the second plurality of nucleic acids is comprised of nucleic acids derived from a non-human source.

7. The array of claim 6 wherein the non-human source is selected from the group consisting of Drosophila, yeast or E. Coli.

8. A set of control nucleic acids for use in comparative genomic hybridization analysis wherein the nucleic acids are derived from a naturally occurring sample that comprises a known aneuploidy.

9. A set of control nucleic acids of claim 8 wherein the known aneuploidy comprises, Turner syndrome (45,X), 47,XXX, Klinefelter syndrome 47,XXY, 45,X/46,XY, 46,XX (male), 46,XX/46,XY(female), 47,XYY, 46,XY/47,XYY, 45,X/46,XY (male), 46,XX/47,XX,del(Yp) (female), or 46,XX/46,XY(female).

10. A set of control nucleic acids of claim 8 wherein the known aneuploidy is Turner syndrome (45,X).

11. A set of control nucleic acids for use in comparative genomic hybridization analysis wherein the nucleic acids are comprised of a mixture of known amounts of cloned nucleic acid segments corresponding to a plurality of chromosomal loci derived from a biological sample.

12. A set of control nucleic acids of claim 11 wherein the biological sample is human.

13. A set of control nucleic acids of claim 11 wherein the biological sample is non-human.

14. A set of control nucleic acids of claim 13 wherein the biological sample is derived from the group of Drosophila, yeast, and E. Coli.

15. A set of control nucleic acids for use in comparative genomic hybridization analysis that comprises a first group of a known amount of nucleic acid segments added to a test sample and a second group of a known amount of substantially identical nucleic acid segments added to a reference sample.

16. A set of control nucleic acids of claim 15 wherein the first and second nucleic acid segments are human.

17. A set of control nucleic acids of claim 15 wherein the first and second nucleic acid segments are non-human.

18. A set of control nucleic acids of claim 17 wherein the non-human nucleic acid segments are derived from the group of Drosophila, yeast, and E. Coli.

19. A method of comparative genomic hybridization analysis that comprises the following steps: (a) providing an array comprising a plurality of nucleic acid target elements, wherein each nucleic acid target element is comprised of a nucleic acid segment that is immobilized to a discrete and known spot on a substrate surface to form an array and the nucleic acid segments comprise a substantially complete first genome of a known mammalian karyotype; (b) providing a first sample, wherein the sample comprises a plurality of genomic nucleic acid segments comprising a substantially complete complement of a first genome labeled with a first detectable label; (c) providing a second sample, wherein the sample comprises a plurality of genomic nucleic acid labeled with a second detectable label, and the genomic nucleic acid sample comprises a substantially complete complement of genomic nucleic acid of a cell or a tissue sample, and the karyotype of the second sample comprises a known abnormal karyotype; (d) contacting the samples with the array of step (a) under conditions wherein the nucleic acid in the samples can specifically hybridize to the genomic nucleic acid segments immobilized on the array; (e) measuring the amount of first and second detectable label on each spot after the contacting of step (d) and determining the karyotype of the first sample by comparative genomic hybridization.

20. The method as recited in claim 19 wherein the karyotype of the second sample comprises a known aneuploidy.

21. The method as recited in claim 20 wherein the aneuploidies includes any of selected from the group comprising, Turner syndrome (45,X), Klinefelter syndrome 47,XXY, 47,XXX, 45,X/46,XY, 46,XX (male), 46,XX/46,XY(female), 47,XYY, 46,XY/47,XYY, 45,X/46,XY (male), 47,XXY male, 46,XX/47,XX,del(Yp) (female), or 46,XX/46,XY(female).

22. The method of claim 20 wherein the known aneuploidy is 47,XXY.

23. The method of claim 19 wherein the array comprises additional nucleic acid target elements derived from a non-mammalian source.

24. The method of claim 23 wherein the non-mammalian source is from the group of Drosophila, yeast, and E. Coli.

25. The method of claim 19 wherein the second sample additionally comprises a known amount of nucleic acid from a separate source.

26. The method of claim 25 wherein the separate source is non-mammalian.

27. The method of claim 26 wherein the non-mammalian source is from the group of Drosophila, yeast, and E. Coli.

28. The method of claim 19 wherein the first and second sample additionally comprise a known amount of nucleic acid from a separate source.

29. The method of claim 28 wherein separate source is non-mammalian.

30. The method of claim 29 wherein the non-mammalian source is from the group of Drosophila, yeast, and E. Coli.

31. The method of claim 26 wherein the known amount of additional nucleic acid generates a standard curve of ratios in a comparative hybridization experiment.

32. The method as recited in claim 19 wherein the first and second detectable labels comprise fluorescent labels.
